(19) United States
(12) Patent Application Publication
(10) Pub. No.: US 2020/0339912 A1
(43) Pub. Date: Oct. 29, 2020

(54) STABILIZATION COMPOSITIONS FOR SCENTED CANDLE FORMULATIONS

(71) Applicant: Vanderbilt Chemicals, LLC, Norwalk, CT (US)

(72) Inventor: Martin PAISNER, Norwalk, CT (US)

(73) Assignee: Vanderbilt Chemicals, LLC, Norwalk, CT (US)

(21) Appl. No.: 16/853,394

(22) Filed: Apr. 20, 2020

Related U.S. Application Data
(60) Provisional application No. 62/838,748, filed on Apr. 25, 2019.

(51) Int. Cl. C11C 5/00; C07D 249/18

(52) U.S. Cl. CPC C11C 5/002; C07D 249/18

(57) ABSTRACT

A stabilization composition for a scented candle formulation comprising:
(A) an aromatic triazole compound of the formula (I)

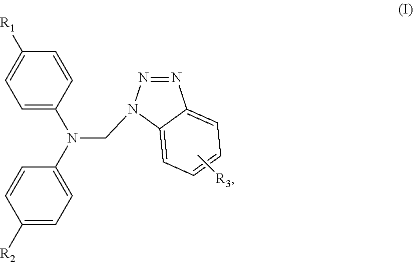

wherein R₁ is a hydrocarbon chain of 4 to 12 carbon atoms, R₂ is a hydrocarbon chain of 4 to 12 carbon atoms, and R₃ is hydrogen or methyl; and
(B) at least one longer wavelength UV light absorber, preferably a 2-(2′-Hydroxyphenyl)benzotriazole.

This application claims the benefit of U.S. Provisional Application No. 62/838,748 filed on Apr. 25, 2019, which is incorporated by reference herein in its entirety.

### BACKGROUND OF THE INVENTION

The invention relates to compositions useful as additives to scented candle formulations which act to reduce color change due to interaction of the fragrance additive with ultra-violet light in the scented candle formulations or products.

### SUMMARY OF THE INVENTION

It is known that compounds added to candle wax preparations for providing a fragrance may cause a negative effect in terms of oxidation and/or chemical changes of the fragrance within the candle caused by exposure to ultraviolet light, resulting in color fade or discoloration. Therefore, it is an object of the invention to provide an additive composition which can mitigate discoloration in scented candle formulations.

As fluorescent light bulbs emit ultraviolet light rays throughout almost the entire UV spectrum, it is beneficial to provide UV absorbers which operate at both the longer and shorter wavelengths within the UV spectrum. For shorter wavelength absorption, a known compound is 2-Hydroxy-4-n-octoxybenzophenone. However, this compound is often in short supply commercially. Therefore, it is advantageous to have a different stabilizing compound which does not need to rely on 2-Hydroxy-4-n-octoxybenzophenone.

Accordingly, the inventive compositions comprise a combination of at least two ultraviolet (UV) light absorber compounds, (A) one of which absorbs light at the shorter wavelengths (100-280 nm) and (B) the second of which absorbs light at the longer wavelengths 280-400 nm). In particular, the invention composition comprises the following two compounds:
(A) a shorter wavelength UV light absorber being an aromatic triazole derivative compound of the formula (I):

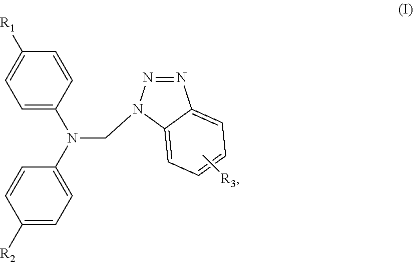

wherein R₁ may be a hydrocarbon chain of about 4 to about 12 carbon atoms, R₂ may be a hydrocarbon chain of about 4 to about 12 carbon atoms, and R₃ may be hydrogen or methyl; and
(B) at least one longer wavelength UV light absorber, preferably a 2-(2′-Hydroxyphenyl)benzotriazole, such as 2-(2′-Hydroxy-5′-methylphenyl)benzotriazole.

In addition to 2-(2′-Hydroxy-5′-methylphenyl)benzotriazole, the stabilizer component (B) longer wavelength UV light absorber, may be one or more of the following 2-(2′-Hydroxyphenyl)benzotriazole compounds: 2-(2′-Hydroxyphenyl)benzotriazoles, for example 2-(2′-hydroxy-5′-methylphenyl)-benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis-(α,α-dimethylbenzyl)-2′-hydroxyphenyl)benzotriazole, 2-(3′-tert-butyl-2′-hydroxy-5′-(2-octyloxycarbonylethyl)phenyl)-5-chloro-benzotriazole, 2-(3′-tert-butyl-5′-[2-(2-ethylhexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylene-bis[4-(1,1,3,3-tetramethylbutyl)-6-benzotriazole-2-ylphenol]; the transesterification product of 2-[3′-tert-butyl-5′-(2-methoxycarbonylethyl)-2′-hydroxyphenyl]-2H-benzotriazole with polyethylene glycol 300; [R—CH2CH2—COO—CH2CH2□2 where R═3′-tert-butyl-4′-hydroxy-5′-2H-benzotriazol-2-ylphenyl, 2-[2′-hydroxy-3′-(α,α-dimethylbenzyl)-5′-(1,1,3,3-tetramethylbutyl)-phenyl]benzotriazole; 2-[2′-hydroxy-3′-(1,1,3,3-tetramethylbutyl)-5′-(α,α-dimethylbenzyl)-phenyl]benzotriazole.

The stabilizer composition may also include one or more optional additives (C) selected from antioxidants, hindered amine light stabilizers, 2-(2H-Benzotriazol-2-yl)-4,6-di-tert-pentylphenol, hindered phenolics and a hindered hydroxyl benzoate. In embodiments, the scented candle formulations may be in the form of a scented candle product. The inventive compositions unexpectedly achieve improved colorfastness upon exposure to UV light sources, such as, for example, fluorescent light bulbs, artificial light, or outdoor sun.

### DETAILED DESCRIPTION OF THE INVENTION

As used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

Stabilization compositions and scented candle formulations and products comprising such stabilization compositions disclosed herein comprise (A) a shorter wavelength UV light absorber being an aromatic triazole derivative compound of the formula (I):

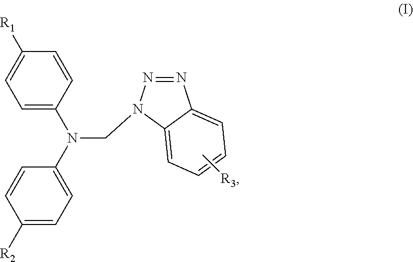

wherein R₁ may be a hydrocarbon chain of about 4 to about 12 carbon atoms, R₂ may be a hydrocarbon chain of about 4 to about 12 carbon atoms, and R₃ may be hydrogen or methyl: and
(B) at least one longer wavelength UV light absorber, preferably a 2-(2′-Hydroxyphenyl)benzotriazole, such as 2-(2′-Hydroxy-5-methylphenyl)benzotriazole.

Preferably, the stabilization compound of Formula I is one where R₃ is methyl group and R₁ and R₂ are octyl groups, namely, 1-[di(4-octylphenyl)aminomethyl]tolutriazole.

Other possible compounds include
1-[di(4-octylphenyl)aminomethyl]benzotriazole,
1-[di(4-butylphenyl)aminomethyl]tolutriazole,
1-[di(4-butylphenyl)aminomethyl]benzotriazole,
1-[di(4-dodecylphenyl)aminomethyl]tolutriazole,
1-[di(4-dodecylphenyl)aminomethyl]benzotriazole,
1-[(4-butylphenyl)(4-octylphenyl)aminomethyl]tolutriazole, and
1-[(4-butylphenyl)(4-octylphenyl)aminomethyl]benzotriazole.

Components (A) and (B) are present in the stabilization composition at weight ratio (A):(B) ranging from about 10:1 to about 1:10, preferably about 5:1 to about 1:5, more preferably from about 3:1 to about 1:3, and even more preferably at from about 1:1 to about 1:3, and most preferably from about 1:1 to about 1:2.5 When additional additives (C) are present in the stabilization composition, the weight ratio of (A) to the combined additives (B) and (C), i.e. (A):[(B)+(C)] is from about 10:1 to about 1:10, preferably bout 3:1 to 1:8, more preferably about 1:1 to 1:8.

As a result of the inclusion of the stabilization composition, a product formed from a candle formulation comprising such composition may improve color stability when compared to commercially existing stabilization formulations, as well as reduce color change due to interaction with UV light. In order to prepare a stabilization formulation, the following method may be employed (i) adding a stabilization composition to the hot, heated, and/or melted wax (hereinafter “the wax”) to prepare or provide a candle formulation (hereinafter “the mixture”), wherein a concentration of components (A)+(B) of the stabilization composition may range from about 0.025 pph to about 1 pph, preferably from about 0.5 pph to about 0.5 pph, and more preferably from about 0.5 pph to about 0.2 pph. The concentration of the stabilization composition containing (A)+(B)+additional additive(s) (C) is present in the wax from about 0.025 pph to about 1.0 pph, preferably about 0.05 to about 0.50 pph, more preferably about 0.10 pph to about 0.40 pph, and even more preferably about 0.19 pph to about 0.34 pph, calculated as parts of the stabilization composition compared to 100 parts by weight of the mixture;
(ii) blending the mixture;
(iii) transferring the blended mixture to a suitable container; and/or
(iv) cooling the blended mixture to ambient temperature.

Optional additional additives (C) may comprise at least one additive selected from at least one hindered amine light stabilizer (hereinafter “HALS”), at least one hindered phenolic, and at least one hindered hydroxyl benzoate. The hindered hydroxyl benzoate may be hexadecyl-3,5-di-t-butyl-4-hydroxybenzoate. The HALS may be, for example, selected from tetrakis(1,2,2,6,6-pentamethylpiperidin-4-yl) Butane-1,2,3,4-tetracarboxylate, or other hindered amine molecules selected from, for example, Bis(1,2,2,6,6-pentamethyl-4-piperidyl) sebacate, 4-hydroxy-2,2,6,6,-tetramethyl-1-piperidine ethanol, and/or poly[[6-[1,1,3,3-tetramethylbutyl)amino]-s-triazine-2,4-dinyl][2,2,6,6,-tetramethyl-4-piperidyl)imino]hexamethylene[2,2,6,6,-tetramethyl-4-piperidyl)imino]. The hindered phenolic may be, for example, selected from tetrakis [methylene-3-(3,5-di-tert-butyl-4-hydroxyphenyl)propionate] methane, [thiodiethylene bis[3-(3,5-di-tert-butyl-4-hydroxyphenyl)proprionate, octadecyl-3-(3,5-di-tert-butyl-4-hydroxyphenyl)proprionate, and tris-(3,5-di-tert-butylhydroxy benzyl)isocyanurate.

At least one benzophenone UV absorber, such as 2-Hydroxy-4-n-octoxybenzophenone, may also be present in the stabilization composition as (A) a shorter wave UV light absorber, in addition to the claimed aromatic tolutriazole derivative.

The scented candle formulations or products may comprise one or more waxes selected from paraffin wax, soybean wax, microcrystalline wax, beeswax, gel, and plant wax. The beeswax may be a byproduct of a honey collection, the gel may be a mixture of polymer and mineral oil, and the plant wax may be based on palm, carnauba, or bayberry. The waxes may be organic and/or may consist of one or more long alkyl chains.

The waxes may contain one or more flavorants and/or fragrances (hereinafter “the fragrance”) designed to achieve a certain pleasing odor or scent. In embodiments, the fragrance may be musk cinnamic aldehyde, ketone, vanillin, and/or ethyl maltol. While many fragrance compounds are aromatics, such as cinnamic aldehyde and vanillin, the fragrance may be selected as one or more in combination of any known fragrance(s) selected from thousands of known chemicals that produce pleasing odor or scent. Each scented candle formulation disclosed herein may contain from about one up to about dozens of different chemicals or fragrances to achieve a specific scent or pleasing scent. Some of these may cause at least one color shift in the products when exposed to UV light.

One particular method disclosed herein may be used to prepare the scented candle formulations and/or products.

In the laboratory, the following procedure may be used to generate the one or more scented candle formulations for lab testing. The scented candle formulations may be generated via a masterbatch preparation. During the masterbatch preparation, the one or more waxes may be weighed out and heated to about 110° F. in an oven to prepare a batch of hot wax. Then, the fragrance(s) may be weighed out and added to the hot wax, and the dyes and/or pigment(s) may be weighed out and added to the hot wax. Subsequently, the batch may be mixed using a hot plate and magnetic stirrer to prepare an un-stabilized candle wax masterbatch.

The following method was used to prepare the scented candle formulations for exposure testing. One or more additives or components of the stabilizing compositions to be tested were weighed out and disposed onto small aluminum pans. The un-stabilized candle wax masterbatch was transferred into a beaker. Then, a magnetic stirrer was disposed into the beaker, the beaker may be placed on a hot plate, and the stabilizing composition was blended into the un-stabilized candle wax masterbatch disposed within the beaker to provide a blended composition of the scented candle formulation. Subsequently, the blended composition was cooled and transferred into sample holders for the exposure testing.

During the exposure testing, samples from the sample holders were placed into “Black Box” apparatus comprising UV fluorescent lamps, in this case “350 Blacklight RG2” bulbs. Then, the UV fluorescent lamps were activated. After about an hour, an intensity of light was tested using a dosimeter that is installed within the apparatus. The intensity was around about 0.9 mw/cm², but other intensity ranges can suffice. A degree of color change in the scented candle formulation was measured using a colorimeter.

### EXAMPLES

Waxes, Fragrances, Pigments

Paraffin and soy waxes containing fragrance, including vanillin, and pigment components were used in the following examples. One set of waxes contained the present stabilization composition disclosed herein and one set contained no stabilizer package.

Additives

Below is a list of the additives used herein:

| | |
|---|---|
| U1 | 2-(2H-Benzotriazol-2-yl)-4,6-di-tert-pentylphenol |
| U2 | 2-(2′-Hydroxy-5′-methylphenyl)benzotriazole |
| U3 | 2-Hydroxy-4-n-octoxybenzophenone |
| U4 | Hexadecyl 3,5-di-t-butyl-4-hydroxybenzoate |
| U5 | Bis(1,2,2,6,6-pentamethyl-4-piperidyl) sebacate |
| V1 | 1-[di(4-octylphenyl)aminomethyl]tolutriazole in 50% Ester* |
| V2 | 1-[di(4-octylphenyl)aminomethyl]tolutriazole 100% Lot 1 |
| V3 | 1-[di(4-octylphenyl)aminomethyl]tolutriazole 100% Lot 2 |
| V4 | 1-[di(4-octylphenyl)aminomethyl]tolutriazole 100% Lot 3 |
| V5 | 1-[di(4-octylphenyl)aminomethyl]tolutriazole in 50% benzyl |
| | benzoate* |
| V6 | 1-[di(4-octylphenyl)aminomethyl]tolutriazole in 40% benzyl |
| | benzoate* |

*Note: amounts of V1, V5 and V6 in the tables below are based solely on the “active” amount, i.e. presence of the tolutriazole molecule, not the total diluted additive.

Testing

Color stability testing is accomplished with the use of the “Black Box” apparatus which utilizes a cabinet containing four “350 UV Black Light RG2 F40T12/350BL” bulbs. These bulbs are inside the cabinet approximately about 18 inches above the sample surface. A dosimeter permanently resides in the apparatus and the intensity of the UV radiation can be directly measure through this dosimeter. Intensity is usually around about 0.9 mw/cm² but with time goes down slightly as the bulbs age.

After exposure, the samples were tested for color change. The CIE L*, a*, b* color coordinates on all samples were measured using a handheld HunterLab Mini Scan EZ 4500L colorimeter. ΔE is the change in color between the samples that was exposed from the initial color.

ΔE_{after time t}=√(ΔL*²+Δa*²+Δb*²)

Reference Samples in the examples below were prepared from a commercially available UV stabilizer package (referred to as “C” in the examples), which is believed to contain a combination of shorter and longer UV wavelength absorbers, including 2-Hydroxy-4-n-octoxybenzophenone, and was added at 0.2 pph.

### Example 1

Starting Materials:

Reference Sample—A paraffin wax containing a commercial UV stabilizer package was selected to compare with the present stabilization composition disclosed herein. The final stabilized paraffin wax was designated “R1”.

Baseline sample—A paraffin wax with no stabilizers was designated “Wax Alone 1”. To this was added stabilizers as defined below in Table 1. Color stability testing was performed as described above. The results are reported below in Table 2.

TABLE 1

| | | Additive Concentrations (pph) | | | | |
|---|---|---|---|---|---|---|
| Sample | C | U2 | U4 | U5 | V1 | Total |
| Wax alone 1 | Balance | Balance | Balance | Balance | Balance | |
| R1 | 0.20 | | | | | 0.20 |
| F1 | | 0.05 | | 0.10 | 0.05 | 0.20 |
| F2 | | 0.05 | 0.10 | | 0.05 | 0.20 |

TABLE 2

| | Exposure (hours) | | |
|---|---|---|---|
| | 68.1 | 122.1 | 168.8 |
| Sample | ΔE | | |
| Wax alone 1 | 22.1 | 23.9 | 26.5 |
| R1 | 15.2 | 20.4 | 22.1 |
| F1 | 11.5 | 14.5 | 15.4 |
| F2 | 14.4 | 17.4 | 18.5 |

The ΔE results clearly show that the stabilizer systems containing the inventive stabilization composition V1 (F1 and F2) suppress color change versus the wax alone and are slightly better than the reference stabilized wax.

### Example 2

Starting Materials:

Reference Sample—A paraffin wax containing a commercial UV stabilizer package was selected to compare with the present stabilization composition disclosed herein. The final stabilized paraffin wax was designated “R2”.

Baseline Sample—A paraffin wax with no stabilizers was designated “Wax Alone 2”. To this was added stabilizers as defined below in Table 3. Color stability testing was performed as described above. The results are reported below in Table 4.

TABLE 3

| | | Additive Concentrations (pph) | | | | |
|---|---|---|---|---|---|---|
| Sample | C | U2 | U4 | U5 | V1 | Total |
| Wax alone 2 | Balance | Balance | Balance | Balance | Balance | |
| R2 | 0.20 | | | | | 0.20 |
| F3 | | 0.05 | | 0.10 | 0.05 | 0.20 |
| F4 | | 0.05 | 0.10 | | 0.05 | 0.20 |

TABLE 4

| | Exposure (hours) | | |
|---|---|---|---|
| | 68.1 | 122.1 | 168.8 |
| Sample | ΔE | | |
| Wax alone 2 | 19.8 | 25.4 | 27.2 |
| R2 | 11.6 | 17.0 | 20.3 |
| F3 | 11.2 | 15.2 | 18.9 |
| F4 | 13.0 | 18.1 | 20.8 |

The ΔE results clearly show that the stabilizer systems containing the inventive stabilization composition V1 (F3 and F4) suppress color change versus the wax alone and are about the same as the reference stabilized wax.

### Example 3

Starting Materials:

Reference Sample—A paraffin wax containing a commercial UV stabilizer package was selected to compare with the present stabilization composition disclosed herein. The final stabilized paraffin wax was designated “R3”.

Baseline Sample—A paraffin wax with no stabilizers was designated “Wax Alone 3”. To this was added stabilizers as defined below in Table 5. Color stability testing was performed as described above. The results are reported below in Table 6.

TABLE 5

| | Additive Concentrations (pph) | | | | |
|---|---|---|---|---|---|
| Sample | C | U2 | U4 | V1 | Total |
| Wax alone 3 | Balance | Balance | Balance | Balance | |
| R3 | 0.20 | | | | 0.20 |
| F5 | | 0.05 | 0.10 | 0.05 | 0.20 |
| F6 | | 0.05 | 0.10 | 0.05 | 0.20 |

TABLE 6

Exposure (hours)

| | |
|---|---|
| 65.8 | 166.70 |

| | |
|---|---|
| Sample | ΔE |

| | | |
|---|---|---|
| Wax alone 3 | 25.2 | 31.4 |
| R3 | 10.4 | 17.1 |
| F5 | 13.6 | 21.2 |
| F6 | 14.8 | 22.3 |

The ΔE results clearly show that the stabilizer systems containing the inventive stabilization composition V1 (F5 and F6) suppress color change versus the wax alone but are not as good as the reference stabilized wax.

### Example 4

Starting Materials:

Reference Sample—A paraffin wax containing a commercial UV stabilizer package was selected to compare with the present stabilization composition disclosed herein. The final stabilized paraffin wax was designated “R4”.

Baseline Sample—A paraffin wax with no stabilizers was designated “Wax Alone 4”. To this was added stabilizers as defined below in Table 7. Color stability testing was performed as described above. The results are reported below in Table 8.

TABLE 7

| | Additive Concentrations (pph) | | | | | | |
|---|---|---|---|---|---|---|---|
| Sample | C | U2 | U5 | V2 | V3 | V4 | Total |
| Wax | Balance | Balance | Balance | Balance | Balance | Balance | |
| alone 4 | | | | | | | |
| R4 | 0.20 | | | | | | 0.20 |
| F7 | | 0.05 | 0.10 | 0.05 | | | 0.20 |
| F8 | | 0.05 | 0.10 | | 0.05 | | 0.20 |
| F9 | | 0.05 | 0.10 | | | 0.05 | 0.20 |

TABLE 8

Exposure (hours)

| | |
|---|---|
| 67.80 | 168.70 |

| | |
|---|---|
| Sample | ΔE |

| | | |
|---|---|---|
| Wax alone 4 | 26.1 | 33.7 |
| R4 | 17.0 | 22.8 |
| F7 | 15.0 | 23.4 |
| F8 | 17.7 | 25.5 |
| F9 | 16.1 | 23.9 |

The ΔE results clearly show that the stabilizer systems containing the inventive stabilization compositions V2 (F7), V3 (F8) or V4 (F9) suppress color change versus the wax alone. Inventive stabilization compositions V2 and V4 are about the same as the reference stabilized wax, while inventive stabilization composition V3 is slightly worse than the reference stabilized wax.

### Example 5

Starting Materials:

Baseline Sample—A paraffin wax was formulated with pigments and fragrances but no stabilizers and was designated “Wax Alone 6”. To this was added stabilizers as defined below in Table 9. Color stability testing was performed as described above. The results are reported below in Table 10.

TABLE 9

| | Additive Concentrations (pph) | | | | |
|---|---|---|---|---|---|
| Sample | U2 | U3 | U4 | V5 | Total |
| Wax Alone 6 | Balance | Balance | Balance | Balance | |
| F10 comp. | 0.05 | 0.05 | 0.10 | | 0.20 |
| F11 comp. | 0.10 | 0.10 | | | 0.20 |
| F12 | 0.10 | | | 0.10 | 0.20 |
| F13 | 0.10 | | | 0.10 | 0.20 |

TABLE 10

Exposure (hours)

| | |
|---|---|
| 66.0 | 113.5 |

| | |
|---|---|
| Sample | ΔE |

| | | |
|---|---|---|
| Wax Alone 6 | 7.6 | 11.4 |
| F10 comp. | 4.1 | 5.4 |
| F11 comp. | 4.4 | 5.7 |
| F12 | 5.4 | 4.0 |
| F13 | 5.7 | 5.9 |

The ΔE results clearly show that the stabilizer systems containing the inventive stabilization composition V5 (F12 and F13) suppress color change versus the wax alone. In addition, the stabilizer systems containing the inventive stabilization composition V5 suppress color about the same as systems containing 2-Hydroxy-4-n-octoxybenzophenone U3 (comparative F10 and comparative F11). This is important because 2-Hydroxy-4-n-octoxybenzophenone is in short supply and not always available. Thus, the inventive stabilization compositions V1 and V5 represent an alternative to U3.

### Example 6

Starting Materials:

Reference Sample—A paraffin wax containing a commercial UV stabilizer package was selected to compare with the present stabilization composition disclosed herein. The final stabilized paraffin wax was designated “Reference 5”.

Baseline Sample—A paraffin wax with no stabilizers was designated “Wax Alone 5.” To this was added stabilizers as defined below in Table 11. Color stability testing was performed as described above. The results are reported below in Table 12.

TABLE 11

| | Additive Concentrations (pph) | | | | | | |
|---|---|---|---|---|---|---|---|
| Sample | C | U2 | U3 | U4 | V1 | V5 | Total |
| Wax | Balance | Balance | Balance | Balance | Balance | Balance | |
| Alone 5 | | | | | | | |
| R5 | 0.20 | | | | | | 0.20 |
| F14 | | 0.05 | 0.05 | 0.10 | | | 0.20 |
| comp. | | | | | | | |
| F15 | | 0.10 | | | 0.10 | | 0.20 |
| F16 | | 0.10 | | | | 0.10 | 0.20 |

TABLE 12

| | Exposure (hours) |
|---|---|
| | 65.7 |
| Sample | ΔE |
| Wax Alone 5 | 19.8 |
| R5 | 15.0 |
| F14 comp. | 15.2 |
| F15 | 16.5 |
| F16 | 14.9 |

The ΔE results clearly show that the stabilizer systems containing the inventive stabilization compositions V1 (F15) and V5 (F16) suppress color change versus the wax alone and are about the same as the reference stabilized wax. In addition, the stabilizer systems containing the inventive stabilization compositions V1 and V5 suppress color about the same as 2-Hydroxy-4-n-octoxybenzophenone U3 (F14 comparative). This is important because 2-Hydroxy-4-n-octoxybenzophenone is in short supply and not always available. Thus, the inventive stabilization compositions V1 and V5 represent an alternative to U3.

### Example 7

Starting Materials:

Baseline Sample—A soy wax was formulated with pigments and fragrances but no stabilizers and was designated “Soya Alone 7”. To this was added stabilizers as defined below in Table 13. Color stability testing was performed as described above. The results are reported below in Table 14.

TABLE 13

| | | | | | V:U2 | | |
|---|---|---|---|---|---|---|---|
| | Additive Concentrations (pph) | | | | wt. | U2 + V | Total |
| Sample | U2 | U3 | U4 | V6 | ratio | pph | pph |
| Soya | Balance | Balance | Balance | Balance | | | |
| Alone 7 | | | | | | | |
| F17 | 0.05 | | 0.10 | 0.04 | 1:1.25 | 0.09 | 0.19 |
| F18 | 0.10 | | 0.20 | 0.04 | 1:2.5 | 0.14 | 0.34 |

TABLE 14

| | Exposure (hours) |
|---|---|
| | 72.7 |
| Sample | ΔE |
| Soya Alone 7 | 12.3 |
| F17 | 10.0 |
| F18 | 9.7 |

The ΔE results clearly show that the stabilizer systems containing the inventive stabilization composition V6 (F17 and F18) suppress color change versus the soya alone.

### Example 8

Starting Materials:

Baseline Sample—A paraffin wax was formulated with pigments and fragrances but no stabilizers and was designated “Wax Alone 8”. To this was added stabilizers as defined below in Table 15. Color stability testing was performed as described above. The results are reported below in Table 16.

TABLE 15

| | Additive Concentrations (pph) | | | | |
|---|---|---|---|---|---|
| Sample | U2 | U3 | U4 | V6 | Total |
| Wax Alone 8 | Balance | Balance | Balance | Balance | |
| F19 comp. | 0.05 | 0.05 | 0.10 | | 0.20 |
| F20 comp. | 0.10 | 0.10 | | | 0.20 |
| F21 | 0.05 | | 0.10 | 0.05 | 0.20 |
| F22 | 0.10 | | | 0.10 | 0.20 |

TABLE 16

| | Exposure (hours) |
|---|---|
| | 68.7 |
| Sample | ΔE |
| Wax Alone 8 | 10.9 |
| F19 comp. | 5.3 |
| F20 comp. | 4.9 |
| F21 | 5.0 |
| F22 | 6.2 |

The ΔE results clearly show that the stabilizer systems containing the inventive stabilization composition V6 (F21and F22) suppress color change versus the wax alone. In addition, the stabilizer systems containing the inventive stabilization composition V6 suppress color about the same as 2-Hydroxy-4-n-octoxybenzophenone U3 (F19 comparative and F20 comparative). This is important because 2-Hydroxy-4-n-octoxybenzophenone is in short supply and not always available. Thus, the inventive stabilization composition V6 represents an alternative to U3.

What is claimed is:

1. A stabilization composition for a scented candle, comprising:
(A) an aromatic triazole compound of the formula (I) 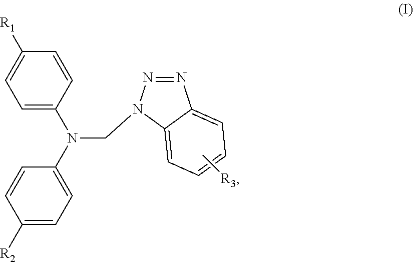
wherein R₁ is a hydrocarbon chain of 4 to 12 carbon atoms, R₂ is a hydrocarbon chain of 4 to 12 carbon atoms, and R₃ is hydrogen or methyl; and
(B) at least one compound which absorbs ultraviolet light rays having wavelength of 280-400 nm.

2. The composition of claim 1, wherein component (B) comprises a 2-(2′-Hydroxyphenyl)benzotriazole.

3. The composition of claim 2, wherein component (B) comprises 2-(2′-Hydroxy-5′-methylphenyl)benzotriazole

4. The composition of claim 1, wherein the weight ratio of (A):(B) is between 3:1 and 1:3.

5. The composition of claim 4, wherein the weight ratio of (A):(B) is between about 1:1 and about 1:3

6. The composition of claim 1, further comprising at least one additional additive (C) chosen from the group consisting of an antioxidant, a hindered amine light stabilizer, a hindered phenolic, and a hindered hydroxyl benzoate.

7. The composition of claim 6, wherein (C) is one or more of tetrakis(1,2,2,6,6-pentamethylpiperidin-4-yl) Butane-1,2,3,4-tetracarboxylate, Bis(1,2,2,6,6-pentamethyl-4-piperidyl) sebacate, 4-Hydroxy-2,2,6,6,-tetramethyl-1-piperidine ethanol, and Poly[[6-[1,1,3,3-tetramethylbutyl)amino]-s-triazine-2,4-dinyl][2,2,6,6,-tetramethyl-4-piperidyl)imino]hexamethylene[2,2,6,6,-tetramethyl-4-piperidyl)imino]; 2-(2H-Benzotriazol-2-yl)-4,6-di-tert-pentylphenol; tetrakis [methylene-3-(3,5-di-tert-butyl-4-hydroxyphenyl)propionate], thiodiethylene bis[3-(3,5-di-tert-butyl 4-hydroxyphenyl)proprionate], octadecyl-3-(3,5-di-tert-butyl-4-hydroxyphenyl)proprionate, tris-(3,5-di-tert-butylhydroxy benzyl)isocyanurate, and hexadecyl-3,5-di-t-butyl-4-hydroxybenzoate.

8. The composition of claim 6, wherein the weight ratio of (A):[(B)+(C)] is about 1:1 to about 1:8.

9. The composition of claim 1, wherein (A) comprises 1-[di(4-octylphenyl)aminomethyl]tolutriazole.

10. The composition of claim 1, wherein the weight ratio of (A):(B) is between about 1:1 and about 1:3; (A) is 1-[di(4-octylphenyl)aminomethyl]tolutriazole;
and (B) is 2-(2′-Hydroxy-5′-methylphenyl)benzotriazole.

11. A scented candle formulation comprising:
a wax component comprising one or more wax compounds and one or more fragrance additives, and
a stabilization composition present in the formulation from about 0.05 pph to about 0.5 pph of the wax component, and wherein the stabilization composition comprises (A) an aromatic triazole compound of the formula (I) 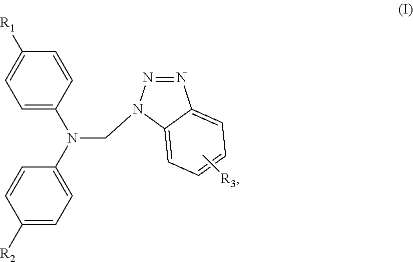
wherein R₁ is a hydrocarbon chain of about 4 to about 12 carbon atoms, R₂ is a hydrocarbon chain of about 4 to about 12 carbon atoms, and R₃ is hydrogen or methyl; and
(B) at least one compound which absorbs ultraviolet light rays having wavelength of 280-400 nm.

12. The formulation of claim 11, wherein component (B) comprises a 2-(2′-Hydroxyphenyl)benzotriazole.

13. The formulation of claim 12, wherein component (B) comprises 2-(2′-Hydroxy-5′-methylphenyl)benzotriazole

14. The formulation of claim 11, wherein the weight ratio of (A):(B) is between 3:1 and 1:3.

15. The formulation of claim 14, wherein the weight ratio of (A):(B) is between about 1:1 and about 1:3

16. The formulation of claim 11, further comprising one or more additional additives (C) chosen from the group consisting of an antioxidant, a hindered amine light stabilizer, a hindered phenolic, and a hindered hydroxyl benzoate.

17. The formulation of claim 16, wherein (C) is one or more of tetrakis(1,2,2,6,6-pentamethylpiperidin-4-yl) Butane-1,2,3,4-tetracarboxylate, Bis(1,2,2,6,6-pentamethyl-4-piperidyl) sebacate, 4-Hydroxy-2,2,6,6,-tetramethyl-1-piperidine ethanol, and Poly[[6-[1,1,3,3-tetramethylbutyl)amino]-s-triazine-2,4-dinyl][2,2,6,6,-tetramethyl-4-piperidyl)imino]hexamethylene[2,2,6,6,-tetramethyl-4-piperidyl)imino]; 2-(2H-Benzotriazol-2-yl)-4,6-di-tert-pentylphenol; tetrakis [methylene-3-(3,5-di-tert-butyl-4-hydroxyphenyl)propionate], thiodiethylene bis[3-(3,5-di-tert-butyl 4-hydroxyphenyl)proprionate], octadecyl-3-(3,5-di-tert-butyl-4-hydroxyphenyl)proprionate, tris-(3,5-di-tert-butylhydroxy benzyl)isocyanurate, and hexadecyl-3,5-di-t-butyl-4-hydroxybenzoate.

18. The formulation of claim 16, wherein the weight ratio of (A):[(B)+(C)] is about 1:1 to about 1:8.

19. The formulation of claim 11, wherein (A) comprises 1-[di(4-octylphenyl)aminomethyl]tolutriazole.

20. The formulation of claim 11, wherein the weight ratio of (A):(B) is between about 1:1 and about 1:3; (A) is 1-[di(4-octylphenyl)aminomethyl]tolutriazole;
and (B) is 2-(2′-Hydroxy-5′-methylphenyl)benzotriazole.
